(19) United States
(12) Patent Application Publication
(10) Pub. No.: US 2007/0259420 A1
(43) Pub. Date: Nov. 8, 2007

(54) Modulating Photoreactivity in a Cell

(75) Inventors: Elias Greenbaum, Knoxville, TN (US); Tanya Kuritz, Kingston, TN (US); Mark S. Humayun, Glendale, CA (US)

Correspondence Address: UT-Battelle, LLC;Office of Intellectual Property, One Bethal Valley Road, 4500N, MS-6258, Oak Ridge, TN, 37831, US

(73) Assignee: UT-BATTELLE, LLC, Oak Ridge, TN (US)

(21) Appl. No.: 11/754,556

(22) Filed: May 29, 2007

Related U.S. Application Data
(62) Division of application No. 10/178,598, filed on Jun. 24, 2002.
(60) Provisional application No. 60/300,349, filed on Jun. 22, 2001.
(60) Provisional application No. 60/300,686, filed on Jun. 25, 2001.

(51) Int. Cl. C12N 5/06

(52) U.S. Cl. 435/325

(57) ABSTRACT

Photoreactivity in a cell is modulated by incorporating an isolated optical trigger on the surface or in the membrane of the cell. Exposure of a cell bearing incorporated optical triggers causes the generation of a measurable physiological signal.

### CROSS REFERENCE TO RELATED APPLICATIONS

The present application claims the benefit of U.S. provisional patent application Nos. 60/300,349 and 60/300,686 respectively filed on Jun. 22, 2001 and Jun. 25, 2001.

### STATEMENT AS TO FEDERALLY SPONSORED RESEARCH

This invention was made with United States government support under grant number DE-ACO5-00OR22725 awarded by the Department of Energy. The United States government has certain rights in the invention.

### FIELD OF THE INVENTION

The invention relates generally to the fields of biophysics, visual science, and medicine. More particularly, the invention relates to methods and compositions for modulating photoreactivity in retinal cells such as bipolar or ganglion cells (GC).

### BACKGROUND

Degenerative retinopathies such as age-related macular degeneration (AMD) or retinitis pigmentosa (RP) are leading causes of blindness world-wide. RP is characterized by progressive photoreceptor degeneration and migration of retinal pigmented epithelial (RPE) cells into the sensory retina. AMD is characterized by deposition of drusen in the RPE and underlying Bruch's membrane and degeneration of the photoreceptors around the macula or central portion of the retina. In both AMD and RP, although the neural wiring connecting the eye to the brain is intact, partial or complete blindness results from loss of photoreceptor cells (e.g., rods and cones). Presently, no treatment for restoring vision lost to RP or AMD is known.

### SUMMARY

The invention relates to the development of compositions and methods for imparting photoreactivity to cells not normally responsive to light. Such photoreactivity is achieved by introducing an optical trigger such as a Photosystem (PS) I reaction center into a target cell, e.g., a retinal cell such as a bipolar cell or a GC. This development might be utilized to reverse blindness caused by damaged or lost photoreceptor cells (e.g., in AMD or RP) by incorporating an optical trigger into bipolar cells or GCs in a blind subject's retina in such a manner that light hitting the cells creates signals (i.e., action potentials) that can be conveyed to the brain. The brain could then interpret these signals as sight.

Accordingly, the invention features method of modulating photoreactivity in a cell. This method includes the steps of (a) providing a cell; and (b) incorporating an isolated optical trigger on the surface or in the membrane of the cell. The latter step can include first preparing a proteoliposome comprising the optical trigger and second contacting the cell with the proteoliposome.

Also within the invention is a cell having an isolated optical trigger incorporated on its surface or in its membrane, and a method of producing a measurable physiological signal (e.g., an action potential) in a nerve cell. The latter method includes the steps of: providing a nerve cell lacking photoreactivity; incorporating an isolated optical trigger on the surface or in the membrane of the cell; and exposing the cell to light.

In the compositions and methods of the invention the optical trigger can be a PS I reaction center; and the cell can be a nerve cell such as a retinal ganglion cell, a retinal bipolar cell, a photoreceptor cell, or a retinoblastoma cell. In some aspects of the invention exposing the optical trigger to light can induce a measurable physiological signal in the cell.

Unless otherwise defined, all technical terms used herein have the same meaning as commonly understood by one of ordinary skill in the art to which this invention belongs. By the phrase “optical trigger” is meant a molecule or functional complex of molecules that can transform light energy into another form of energy, especially chemical energy. When referring to a molecule or complex of molecules such an optical trigger, the term “isolated” means separated from components that naturally accompany such molecule or complex of molecules. Typically, an optical trigger is isolated when it is at least 30% (e.g., 40%, 50%, 60%, 70%, 80%, 90%, and 100%), by weight, free from the proteins or other naturally-occurring organic molecules with which it is naturally associated. A chemically-synthesized or otherwise man-made molecule or complex of molecules is also considered “isolated.” Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including any definitions will control. In addition, the particular embodiments discussed below are illustrative only and not intended to be limiting.

Other features and advantages of the invention will be apparent from the following detailed description, and from the claims.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 is a schematic diagram of a PS I reaction center in the photosynthetic membrane of a chloroplast.

FIG. 2 is a schematic illustration of a method of incorporating an optical trigger onto a cell shown in two stages. Stage 1 shows the formation of PS I-proteoliposomes and incorporation in a cultured retinal cell. Stage 2 shows PS I-proteoliposome stabilization, modification, and incorporation in a retinal neuron.

FIG. 3 is a highly schematic illustration of PS I-assisted depolarization and hyper-polarization of a cell incorporated with a PS I reaction center.

FIG. 4A is a graph showing the response to light of cultured retinal cells incorporated with PS I-proteoliposomes.

FIG. 4B is a graph showing the response to light of cultured retinal cells incorporated with control proteoliposomes devoid of PS I reaction centers.

### DETAILED DESCRIPTION

The invention relates to development of novel compositions and methods for modulating photoreactivity in cells. Utilizing the invention, photoreactivity can be imparted to cells that are normally unreactive to light (e.g., visible light).

The invention draws from two seemingly diverse areas of biophysics: (1) photosynthesis, i.e., the transduction of light energy into stored chemical energy by green plants and (2) visual phototransduction, i.e., the transduction of light energy by a photoreceptor into a nerve impulse. In both photosynthesis and visual phototransduction, photoreactivity is mediated through optical triggers incorporated in cell membranes. In the invention, photoreactivity is modulated in a cell by incorporating an isolated optical trigger in the membrane of the cell. This development might be utilized to reverse blindness caused by damaged or lost photoreceptor cells (e.g., in AMD or RP) by incorporating an optical trigger into bipolar cells or GCs in a blind subject's retina in such a manner that light hitting the cells creates signals (i.e., action potentials) that can be conveyed to the brain. The brain could then interpret these signals as sight.

The below described preferred embodiments illustrate adaptations of these compositions and methods. Nonetheless, from the description of these embodiments, other aspects of the invention can be made and/or practiced based on the description provided below.

### Photon Absorption in Photosynthesis and Vertebrate Vision

In both photosynthesis and visual phototransduction, the process of photoreactivity begins when visible photons (400-700 nm) are absorbed by pigment molecules anchored to proteins. In photosynthesis by green plants, absorption of light occurs in specialized reaction centers known as Photosystems I and II (PS I and PS II). The reaction centers are contained in light-sensitive organelles in plant cells known as chloroplasts. Chloroplasts contain orderly stacks of photosynthetic membranes in which the PS I reaction centers form functional complexes made up of 14-15 proteins. (Chitnis, P. R., Ann. Rev. Plant Physiol. Plant Mol. Biol. 52: 593-626, 2001).

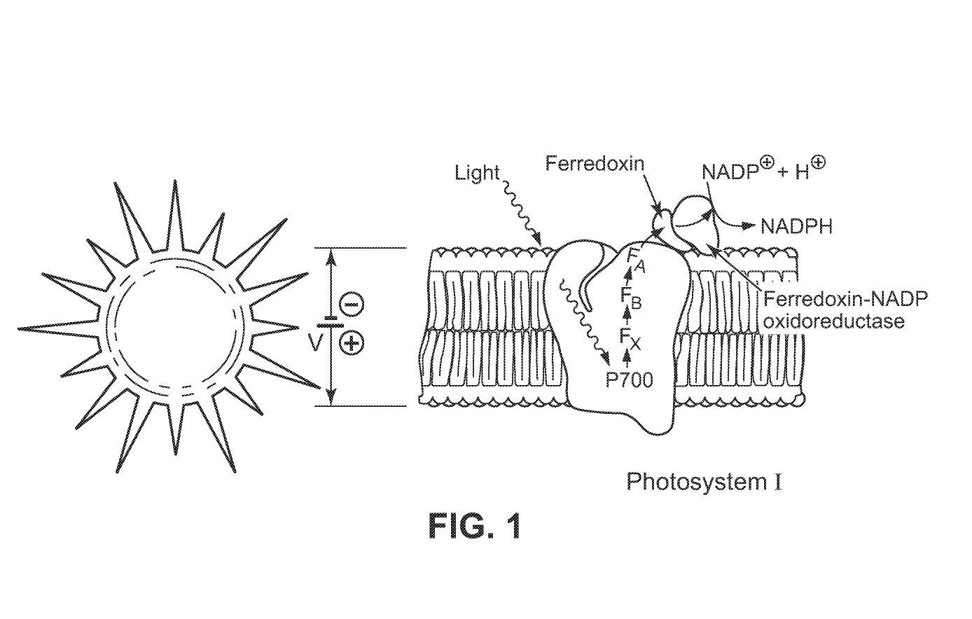

The overall function of PS I reaction centers is to harvest photons and use their energy for electron transfer through a series of redox centers. The end result of this process is the triggering of a rapid charge separation and conversion of light energy into an electric voltage across the reaction centers. This generates an electrical potential difference of at least 1 V across the photosynthetic membrane. As shown in FIG. 1, PS I reaction centers are integral membrane protein complexes that span photosynthetic membranes. The pigments in PS I reaction centers that capture light are located in the antenna complex. When these pigments absorb light, the photon energy is transferred through the antenna pigment molecules to the P700 reaction center, made up of a chlorophyll dimer. In the P700 center, the excitation energy is trapped and used for energizing an electron, which is transferred to an acceptor chlorophyll a molecule (A₀). Electrons from the reduced A₀ are subsequently transferred through a chain of redox centers designated Fₓ, F_{B} and F_{A}. The terminal electron donor then reduces ferredoxin, which in turn provides energy in a variety of chloroplast reactions including NADPH production, (P. R. Chitnis, Ann. Rev. Plant Physiol. Plant Mol. Biol. 52: 593-626, 2001).

In some embodiments, the invention uses isolated PS I reaction centers to instill into non-photoreactive cell types the ability to generate electrical potentials in response to light. For example, incorporation of a PS I reaction center in a retinal cell such as a bipolar cell or a GC could be used to generate a light-induced action potential that could mimic the response of a photoreceptor to light. The basis for this approach is the fact that visual phototransduction, like photosynthesis, requires a change in voltage across a cell membrane.

In particular, visual phototransduction in the retina begins when light strikes the photoreceptors, i.e., the rods and cones. Whereas the antenna pigments are responsible for light capture in photosynthesis, the pigment in vision is opsin (rhodopsin in rods), complexed with a chromophore, i.e. 11-cis-retinal. Opsin molecules are contained in light-sensitive portions of the photoreceptor cells known as outer segments. Similar to chloroplasts having stacks of photosynthetic membranes with inserted PS I reaction centers, outer segments contain stacks of membranous discs in which opsin is inserted as an integral membrane protein. When light strikes the retina, photon absorption by 11-cis-retinal triggers isomerization of the molecule to all-trans-retinal. This in turn leads to a cascade of biochemical reactions that culminates in the closing of cation-specific channels in the plasma membrane of the outer segment, leading to a hyperpolarization of the plasma membrane. (In hyperpolarization, the normally negative interior membrane potential becomes even more negative.) The light-induced hyperpolarization is then transmitted via the plasma membrane from the outer segment to the synaptic terminus of the photoreceptor, where it is sensed and propagated to other neurons of the retina. (Stryer, L., Biochemistry, W.H. Freeman and Co., New York, N.Y., 1995). Thus detection of light by photoreceptors results in the generation of a nerve impulse that is transmitted to other neurons in the retina.

A single photon absorbed by a dark-adapted rod photoreceptor leads to a hyperpolarization of about 1 mV. Accordingly, the creation of an action potential (i.e., hyperpolarization or depolarization) of this magnitude in a cell, such as a retinal bipolar cell or a GC, downstream of the photoreceptors within the retinal neuronal circuitry might be predicted to mimic the effect of an action potential generated by a photoreceptor.

### Optical Triggers

The invention provides compositions and methods for imparting photoreactivity to cells not normally responsive to light. Such photoreactivity is achieved by expressing an optical trigger such as a Photosystem (PS) I reaction center in a target cell. By introducing an optical trigger into bipolar cells or GCs of a retina, these cells can be made photoreactive.

Several different types of optical triggers that can be used in the invention are known. These include rhodopsin, rhododsin-like molecules such as bacteriorhodopsin, Photosystem I and II (PS-I and PS-II) reaction centers, and artificial photosynthetic reaction centers (see, e.g., “Mimicking Bacterial Photosynthesis,” D. Gust, T. A. Moore and A. L. Moore, Pure & Appl. Chem., 70, 2189-2200, 1998). In preferred embodiments described in the examples section, PS I reaction centers are isolated from plant chloroplasts and transferred to retinal cells to impart photoreactivity to these cells.

Optical triggers for use in the invention can be isolated according to known methods. For example, PS I reaction centers can be isolated from photosynthetic membranes as described by Lee, J. W. et al., Biophys. J., 69:652-659, 1995. Isolated PS I reaction centers are preferred in the invention as these have been shown to act as robust molecular photovoltaic devices that are amenable to ex vivo manipulation and can remain stable following periods of storage of up to 4 months in a vacuum desiccator (Greenbaum, E., Science, 230:1373-1375, 1985; Greenbaum, E., J. Phys. Chem. 92: 4751-4754, 1988; Lee, J. W. et al., Biophys. J. 69:652-659, 1995; Lee, I. et al., Proc. Nat'l. Acad. Sci. USA, 92:1965-1969, 1995; Lee, I. et al., Phys. Res. Lett., 79:3294-3297, 1997; Lee, J. W. et al., J. Phys. Chem. B, 102:2095-2100, 1998; Lee, I. et al., J. Phys. Chem. B, 104:2439-2443, 2000; Millsaps, J. F. et al., Photochem. Photobiol. 73:630-635, 2001).

### Incorporation of Optical Triggers Into Proteoliposomes

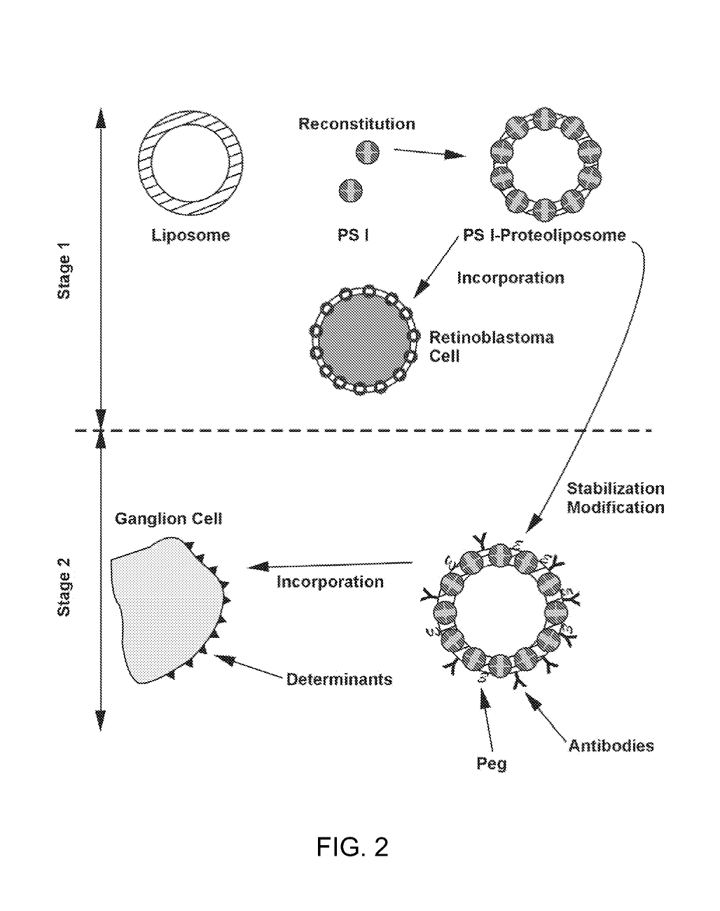

Referring to FIG. 2, to facilitate their incorporation into cells, optical triggers are reconstituted in lipid vesicles (i.e., liposomes) to form proteoliposomes. Several different methods of making liposomes and proteoliposomes are known. Any of these that are suitable for producing the proteoliposomes of the invention may be used. Methods for preparing liposomes particularly suited for a specific application have been previously described (e.g., Gregoriadis G., ed., Liposome Technology, Vol. III. Targeted Drug Delivery and Biological Interaction. CRC Press, Boca Raton, Fla., 1984; Green, R. and Widder, K. J., eds., Methods in Enzymology Vol. 112, Drug and Enzyme Targeting, Part B, Academic Press, San Diego, Calif., 1985; Green, R. and Widder, K. J., eds., Methods in Enzymology Vol. 149, Drug and Enzyme Targeting, Part B, Academic Press, San Diego, Calif., 1987.) For compatibility of liposomes with mammalian cell membranes, PC/cholesterol lipid mixtures are used. A preferred embodiment of the invention employs protocols for preparation of human cell-targeted liposomes (Fonseca M. J. et al., Biochim. Biophys. Acta, 1419:272-282, 1999; Tseng Y. L. et al., Int. J. Cancer 80: 723-730, 1999.)

Methods are known for reconstituting transmembrane proteins and complexes including PS I reaction centers into proteoliposomes (e.g. Kiefer H. et al., Biochemistry 35:16077-16084, 1996; Cladera J. et al., J. Bioenerg. Biomembr., 28:503-515, 1996; Kruip, J. et al., J. Biol. Chem., 274:18181-18188, 1999). Isolation of PS I reaction centers, which are a transmembrane complexes, typically requires membrane solubilization with detergents. Reconstitution of membrane proteins into proteoliposomes can be achieved by step-wise solubilization of preformed liposomes, and membrane protein incorporation at different stages of the solubilization process (Paternostre M. T. et al., Biochemistry, 27:2668-2677, 1988). Cholesterol content is known to affect membrane permeability making it prone to membrane protein insertions (Raffy S, and J. Teissie, Biophys J., 76: 2072-2080, 1999). An optimized protocol developed for PS I reconstitution employs destabilization of preformed soybean PC/phosphatidic acid liposomes by saturating amounts of detergents prior to the addition of PS I complexes (Cladera J. et al., J. Bioenerg. Biomembr., 28:503-515, 1996; Kruip, J. et al., J. Biol. Chem., 274:18181-18188, 1999). The nature of the detergent may affect orientation of the liposome-incorporated protein (Knol J. et al., Biochemistry, 37:16410-16415, 1998). PS I-proteoliposomes thus prepared are subsequently purified by size-exclusion chromatography.

The process of PS I insertion can be assessed in real time in liquid samples using tapping-mode atomic force microscopy (AFM) (Hansma, H. G. and J. H. Hoh, Annu. Rev. Biophys. Biomol. Struct., 23:115-139, 1994). Liposome-associated fluorescence due to incorporation of PS I reaction centers is measured using a fluorometer, e.g. a Waltz PAM fluorometer equipped with a P700 absorption spectrometer accessory.

### Incorporation of Optical Triggers Into Cells

Referring again to FIG. 2, to modulate photoreactivity in a cell, optical triggers are incorporated into cells such as cultured retinal cells (Stage 1), or into retinal neurons in situ (Stage 2). In a preferred version of this method, the target cell is contacted with proteoliposomes that include an optical trigger such as a PS I reaction center. This contacting step is performed under conditions in which the plasma membrane of the cell fuses with the proteoliposomes. After fusion, the target cell plasma membrane contains the optical trigger.

The invention can be used to impart photoreactivity to cells grown in culture or to cells targeted in situ. Although methods of the invention are believed to be useful for modulating photoreactivity in all or almost all cell types, in order to further methods for restoring sight to a blind subject, nerve cells such as retinal cells (e.g., bipolar cells, GCs, or photoreceptor cells with defective optical triggers) are preferred targets for introducing optical triggers. Exemplary retinal cells that can be cultured in vitro include retinoblastoma cells and GCs. Methods are well known for culturing retinoblastoma cells (e.g., Abmad, I., and T. M. Allen, Cancer Res. 52:4817-4820) and for preparing cultures of purified GCs (e.g. Otori Y. et al., Invest. Opthalmol. Vis. Sci. 39:972-981, 1998; Shen S. et al., Neuron 23:285-295, 1999.) A human retinoblastoma cell line such as WERI-Rb (ATCC HTB-169) may be advantageously used for in vitro applications because its biochemistry and physiology have been extensively characterized.

In certain variations of the invention it is desirable to modify the proteoliposomes after they have been made. For example, the proteoliposomes of the invention can be stabilized using polyethylene glycol (PEG) according to known methods. Optimization of saturation of the cell membranes with PS I reaction centers and stabilization of insertion of the PS I may be achieved through stabilization of the liposomes. It is known that liposome stability in tissues and fluids depends on steric parameters (Torchilin V. P. et al., In: Proc. 20^{th} Int. Symp. Control. Release Bioactive Mat., Controlled Release Society, Inc. Washington, D.C., 194-195, 1993). Proteoliposomes of the invention are stabilized by previously described methods, e.g. lipid derivatization by poly(ethyleneglycol) (Torchilin V. P. et al., In: Proc. 20^{th} Int. Symp. Control. Release Bioactive Mat., Controlled Release Society, Inc. Washington, D.C., 194-195, 1993; Kirpotin, D. et al., Biochemistry, 36:66-75, 1997; Lopes de Menezes D. E., et al., Cancer Res. 58:3320-30, 1998; Gregoriadis, G. and A. T. Florence, Drugs, 45:15-28, 1993).

In addition to stabilization of the proteoliposomes, it may be desirable to perform further modifications, for example to assist in targeting the proteoliposomes to particular cell types (e.g., GCs or bipolar cells). As shown in FIG. 2, the proteoliposomes can be conjugated with molecules (e.g., antibodies) that specifically bind a particular target molecule (e.g., an antigen) on the surface of the target cells. Methods for conjugating molecules such as antibodies to proteoliposomes are known (Torchilin V. P. et al., In: Proc. 20^{th} Int. Symp. Control. Release Bioactive Mat., Controlled Release Society, Inc. Washington, D.C., 194-195, 1993; Torchilin, V. P., J. Mol. Recognit., 9:335-346, 1996; Torchilin, V. P., Mol. Med. Today, 2:242-249, 1996; Torchilin V. P. et al., Biochim. Biophys. Acta., 1279:78-83, 1996). In preferred embodiments in which GCs or bipolar cells are the targets for PS I proteoliposome delivery, advantage is taken of specific target molecules characteristic of the particular cell types. For example, expression of cadherin-7, (a Type II cadherin), is restricted to neurons in the inner neuroblast and developing GC layers of the mouse retina (Faulkner-Jones et al., 1999). Thy-1 antigen is a well known specific marker of GCs in rodent retinas (Barnstable, C. J. and U. C. Drager, Neuroscience 11:847-855, 1984). Target molecules such as cadherin 7 and Thy-I can be used advantageously for targeting proteoliposomes to GCs by incorporating into the proteoliposomes antibodies that bind to these target molecules. As an example, 2D12, a monoclonal antibody directed against Thy-I antigen (Barnstable, C. J. and U. C. Drager, Neuroscience 11:847-855, 1984) can be used to make proteoliposomes targeted to GCs.

The invention can also be used to target photoreceptor cells. This approach may be desirable in conditions in which the photoreceptors are intact but not functional. For this purpose, photoreceptor-specific antigens can serve as targets, including PHR1, which is expressed in photoreceptors and GCs (Xu S. et al., J. Biol. Chem., 274:35676-35685, 1999), and tubby-like proteins (Ikeda, S. et al., Invest. Opthalmol. Vis. Sci., 40:2706-2712, 1999). If a target cell is provided in a mixture of different cells, proteoliposomes conjugated with an antibody that specifically binds an antigen on the target cell are preferentially directed to fuse with the target cells. This method can be performed in vitro (e.g., in an in vitro culture of cells) or in vivo (e.g., in the eye of a subject). A major objective of the invention is to provide for liposome-assisted insertion of optical triggers such as PS I reaction centers into one or several of the ensembles of cells (especially bipolar cells and GCs) that participate in optical signaling and information transfer to the brain via the optic nerve, and thereby to restore light responsiveness. Of the retinal neurons in situ, bipolar cells might be the preferred targets, as they are situated at a relatively proximal location in the visual pathway, i.e., one step beyond the photoreceptors. Thus activation at the bipolar cell level might permit a more physiologic processing of the signal at higher retinal levels. Alternatively, incorporation of optical triggers into GCs would also permit transmission of the light-generated impulse to the visual cortex of the brain.

In a preferred method of introducing optical triggers into retinal neurons in situ, proteoliposomes incorporating the optical triggers are prepared in a mixture suitable for intraocular injection (e.g., sterilized and in an ocularly acceptable carrier). The proteoliposome mixture is then administered to a subject so that the proteoliposomes are delivered to the retina. For example, the mixture can be intraocularly injected or delivered to the retina using a cannula.

### PS I Activation of a Nerve Cell

The invention also provides methods of using optical triggers such as PS I reaction centers to generate a measurable physiological signal (e.g., the generation of an electrical potential) in a nerve cell. It has been recently demonstrated that isolated PS I can function as nanoscale photovoltaic devices. (Lee, I. et al., J. Phys. Chem. B, 104:2439-2443, 2000.) The light-induced photovoltaic property of isolated PS I reaction centers generates sufficient voltage (at least 1V) to trigger an action potential in neural cells. Advantageously in this application, PS I reaction centers can repeatedly capture photons and generate a voltage because they can repolarize without the need for energy or complex molecular reorientation (P. R. Chitnis, Ann. Rev. Plant Physiol. Plant Mol. Biol. 52: 593-626, 2001), as is required for the optical trigger in the vertebrate retina, i.e. rhodopsin.

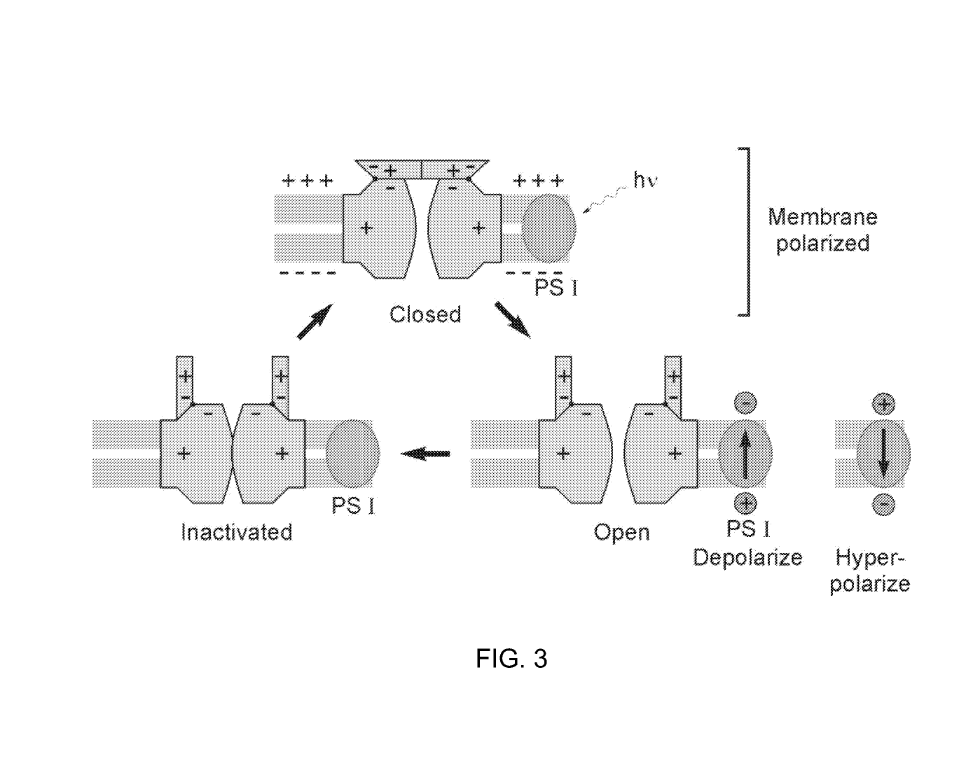

Referring now to FIG. 3, a schematic diagram of a method of PS I-assisted generation of an electrical potential in a neural cell includes the step of inserting a PS I reaction center into the plasma membrane of the neural cell in proximity to a voltage-gated ion channel. Following photon absorption, the PS I reaction center triggers a vectorial charge separation by electron donation from P700 to the FAB electron acceptor complex, as described above. This in turn can regulate the opening and closing of the ion channel, resulting in the generation of a electrical potential. FIG. 3 illustrates one possible way in which PS I reaction centers can trigger an action potential. The interior of most cells (including neurons) has a high concentration of K⁺ and a low concentration of Na⁺. These ionic gradients are generated by an ATP-driven pump. In the resting state (ion channel closed, top figure) the membrane of an axon is much more permeable to K⁺ than to Na⁺ and so the membrane potential is largely determined by the ratio of the internal to the external concentration of K⁺. A nerve impulse, or action potential, is generated when the membrane is depolarized beyond a critical threshold. This occurs when the ion channel (channel open, bottom right) opens under the influence of the light-induced voltage generated by the PS I reaction center. As a result of the inflow of additional cations such as Na or Ca⁺⁺, the membrane potential becomes positive within about a millisecond, and attains a value of about +30 mV before turning negative again. This amplified depolarization, triggered by PS I, is propagated along the nerve terminal. While this mechanism for generation of an action potential is generally accepted by membrane physiologists, other physical and chemical phenomena may also occur to influence the movement of ions across the cell membrane.

By selecting proteoliposomes with all or most of the optical triggers oriented in a particular direction, the optical triggers can be incorporated onto the target cell surface in a functional orientation. Neural cells with functionally oriented optical triggers such as PS I reaction centers can be either hyperpolarized or depolarized, depending upon the orientation of the PS I reaction centers in the plasma membrane (FIG. 3). This light-induced change in charge can be used for example to trigger an action potential.

Methods are known for insertion of optical triggers into proteoliposomes in a particular orientation. In PS I-proteoliposomes, molecular orientation of reconstituted PS I reaction centers is determined by electrostatic interactions. In liposomes formed of soybean lecithin membranes reconstituted with PS I reaction centers, negative charging of the liposome interior was observed, consistent with unidirectional orientation of the reaction centers with the primary electron donor located on the external surface of the liposomes (Gourvoskaya K. N. et al., FEBS Lett., 414:193-196, 1997). Similar unidirectional orientation of PS I reaction centers was shown in PC/phosphatidic acid liposomes by measurement of an inward H⁺ flux (Cladera J. et al., J. Bioenerg. Biomembr., 28:503-515, 1996). Uniformity of orientation of PS I reaction centers in liposome membranes depends on the liposome charge, which may also play a role in proteoliposome stabilization (Hara M. et al., Biosci. Biotechnol. Biochem., 61:1577-1579, 1997). Also, the nature of the detergent used in the reconstitution of transmembrane proteins into liposomes may affect the orientation of the liposome-incorporated protein (Knol J. et al., Biochemistry 37:16410-16415, 1998).

The orientation of PS I reaction centers in proteoliposomes can be determined by D-D mapping in solution (Heinz, W. F., J. H. Hoh, Biophys J., 76:528-538, 1999). Upon light absorption, PS I generates a voltage of l V, in which case a significant surface charge density change on the liposome surface is observed. The orientation of PS I in proteoliposomes can also be determined by Kelvin probe force microscopy in air (Wiegrabe W. et al., J. Microsc. 163:79-84, 1991). The latter method can be used to measure the light-induced potential generated by immobilized PS I on gold (111) surfaces with a I mV standard deviation.

### EXAMPLES

### Example 1

### Preparation of Liposomes

Experimental Protocol: A synthetic membrane was prepared from a saturated phosphatidylcholines (PC)-cholesterol mixture 1:1, 5:1, and 10:1 mole fractions, as described for preparation of human cell-targeted liposomes (Fonseca M. J. et al., Biochim. Biophys. Acta., 1419:272-282, 1999; Tseng Y. L. et al., Int. J. Cancer 80: 723-730, 1999). The lipids were dissolved in chloroform and the solution was dried under vacuum to remove traces of chloroform. The dry lipid film was hydrated to a final concentration of 20 mg lipids ml⁻¹ in 25 mM Tris-HCl, pH 7.8, 1 mM dithiothreitol, 10 mM MgCl₂, and 10 mM NaCl, essentially as described (Kruip, J. et al., J. Biol. Chem., 274:18181-18188, 1999). Hydrated lipids were then subjected to sonication for 10-30 min until clear solutions were obtained. The liposomes were stored at 4° C. and were extruded under pressure using, a Lipex Liposome Extruder.

### Example 2

### PS I Reconstitution into Proteoliposomes

Experimental Protocol: Liposomes were prepared essentially as described (Kruip, J. et al., J. Biol. Chem., 274:18181-18188, 1999). n-Octyl b-D-glucopyranoside was added to concentrations of 1, 2, 5, and 10% (w/v), and the lowest concentration was selected that allowed complete solubilization of the liposomes, PS I reaction centers were isolated as previously described. Briefly, PS I reaction center/core antenna complexes containing about 40 chlorophylls per photoactive P700 (PS I-40) were isolated from spinach thylakoids using detergent (Triton-X 100) solubilization and hyroxylapatite column purification (Lee, J. W. et al., 1993), followed by elution in a buffer containing 0.2M phosphate, pH 7.0, and 0.05% Triton-X 100. The characteristics of the PS I reaction centers were confirmed by absorption spectroscopy (maxima at 440 nm and 672 nm) and by P700 oxidation kinetic spectroscopy in the presence of 20 mM sodium ascorbate and 0.5 mM methyl viologen. P700 oxidation measurements were performed with a Walz spectrometer that measured the kinetic profile of the light-induced differential absorption between 810 and 860 nm. In the presence of sodium ascorbate, the reduction kinetics of P700+ were biphasic: 24 s⁻¹ for reduction by F_{AB}⁻ and 0.1 s⁻¹ by ascorbate. This functional assay did not require PS I reaction centers to be first incorporated in liposomes or cells.

Isolated PS I reaction centers were added to the solubilized liposomes at chlorophyll: lipid ratios between 50 and 200 (w/w). After incubation (5 min) at room temperature, the detergent was removed from the suspension by hourly addition of SM-2 BioBeads (80-60 mg ml⁻¹). PS I-proteoliposomes were removed and purified by size exclusion chromatography on a Sepharose CL-2B column equilibrated with hydration buffer containing 25 mM Tris-HCl, 10 mM MgCl₂, 10 mM NaCl and 11M DTT. Proteoliposome suspensions were then sterilized by filtration through 0.22 μm sterilization filters (Nalgene). PS I content in the PS I— proteoliposomes was measured by phosphorus assay used to estimate lipid 11 content (Fiske, C. H. and Y. Subbarow, J. Biol. Chem. 66: 375-400, 1926), by light-induced differential absorption spectroscopy, and by fluorescence intensity. PS1 proteoliposome size was measured by light scattering.

As an alternative method of proteoliposome preparation, a buffer containing 25 mM Tris-HCl, pH 7.8, 10 mM MgCl₂, 10 mM NaCl, 1 mM DTT was prepared. Hydrogenated soy phosphotidylcholine (HSPC) (76 mg) and cholesterol (4 mg) were dissolved in 1 ml chloroform at a HSPC: cholesterol molar ratio of 10:1. The HSPC/cholesterol solution was then divided into aliquots, dried under a nitrogen stream and stored in a dessicator. The lipid film in each tube was subsequently hydrated to a final concentration of 20 mg/ml in 0.5 ml of buffer. The suspension was then sonicated in a sonicator bath under nitrogen, twice for 5 minutes, with a 15 minute interval in between. An additional 1.5 ml of buffer was added and incubation proceeded for 1.5 h, followed by sonication for 5 minutes. Following transfer into 1,5-ml polypropylene tubes and centrifugation in an Eppendorf microcentrifuge for 2 min at 14,000 rpm, the supernatant was divided into 2×900 μl aliquots and placed in Corex centrifuge tubes with stir bars. Triton X-100 stock solution (10% Triton-X in H₂O, 90 ml) was added to each of the aliquots of liposome suspension and stirred for 1 hr.

PS I reaction centers were then added to the solubilized liposomes, e.g. about 2 mg of PS I to 20 μg of liposomes, and incubated at room temperature for 30 min with agitation. The absorption spectrum of the PS I preparation was taken and peak values and wavelengths were measured. The proteoliposomes thus prepared were then reconstituted by gel chromatography on a Sepharose 6B column (12 ml) equilibrated with buffer. A maximum volume of 1.2 ml was loaded onto the column. A void volume of approx. 3.8-4.0 ml was collected and removed, then fractions approximately 1 ml in volume were collected in Eppendorf tubes. Spectra for each fraction were measured and those fractions containing PS I-proteoliposomes were pooled, spun down and resuspended in 1 ml of buffer.

The orientation of PS I reaction centers in proteoliposomes was determined by D-D mapping in solution (Heinz, W. F., J. H. Hoh, Biophys J., 76:528-538, 1999). To perform real-time AFM diagnostic studies of the PS I insertion process, the liposomes were kept in a stationary state on a flat substrate. For gold (111)-coated substrates, a thiol-bearing reagent was used as a cross-linker for liposomes and a gold surface (Wagner P. et al., Biophys J., 70:2052-2066, 1996). Glass substrates were activated with a silane regent (Yoshino, 1994) and made functional toward reactive groups of PS1 proteoliposomes. Solutions of liposomes or PS I-proteoliposome were directly injected into the AFM liquid cell and self-immobilized onto the substrate without rinsing loosely bound liposomes off the substrate.

### Example 3

### Retinal Cultures Used For Incorporation of PS I-Proteoliposomes

Retinoblastoma cultures. WERI-Rb−1 human retinoblastoma cell line (ATCC HTB-169) were maintained by passage and growth to cell densities between 10⁵ and 10⁶ ml⁻¹ in suspension cultures in RPMI-1640 medium supplemented with 10% FBS at 37° C. in a humidified incubator with 5% carbon dioxide. The cultures were prepared essentially as described (Ahmad, I. and T. M. Allen, Cancer Res., 52:4817-4820, 1992; Lopes de Menezes D. E et al., Cancer Res. 58:3320-30, 1998. 96). The cells were washed with fresh growth medium by low-speed centrifugation at room temperature and cell density was measured. 200 μl aliquots containing 10⁶ cells were plated in triplicate in 6-well culture plates and left to recover for 2 h under growth conditions.

GC cultures. A purified culture a rat retinal GCs is prepared according to previously established techniques (Otori Y. et al., Invest. Opthalmol. Vis. Sci., 39:972-81, 1998; Pereira, S. P. and E. G. Araujo, Braz. J. Med. Biol. Res. 30: 1467-1470, 1997; Taschenberger H. et al., J. Neurosci., 19: 3353-3366, 1999; Shen S. et al., Neuron., 23:285-295, 1999). Briefly, following euthanization with intraperitoneal sodium pentothal and enucleation, neural retinal tissue is isolated from the eyes of 6-8 day old Long Evans rats. A retinal cell suspension is prepared by enzymatic dissociation as follows. The neural retinas tissue is incubated at 370C for 30 minutes in a solution of papain (15 U/ml) and collagenase (70 U/ml) in Hanks' balanced salt solution containing 0.2 mg/ml bovine serum albumin (BSA) and 0.2 mg/ml DL-cysteine. The tissue is then triturated sequentially through a narrow-bore Pasteur pipette in a solution containing 2 mg/ml ovomucoid, 0.004% deoxyribonuclease, and 1 mg/ml BSA. The cells are centrifuged at 600 rpm for 5 minutes, rewashed in a solution containing 10 mg/ml each of ovomucoid and BSA, then resuspended in 0.1% BSA in phosphate-buffered saline (PBS).

For separation of the GCs from other cell types in the suspension, use is made of specific antibodies, i.e. 2G12 and MAC1. 2G12 is a monoclonal ascites IgG antibody against rat Thy-1, a cell surface marker specific for GCs (Barnstable, C. J. and U. C. Drager, Neuroscience 11:847-855, 1984). MAC1 is a monoclonal supernatant antibody against mouse macrophages. Polypropylene tubes (50 ml) are incubated at 40 C overnight with 2 ml PBS containing the primary antibodies diluted as follows: 2G12 (1:100) and MAC1 (1:10). Tubes are subsequently washed three times with 3 ml PBS. To prevent non-specific cell binding to the panning tubes, 4 ml PBS containing 0.1% BSA is applied to the coating area.

The retinal cell suspension is incubated in MAC1-coated tubes at room temperature 30 minutes, and gently rotated to allow access of all cells to the surface of the coating area. Non-adherent cells are removed and placed in 2G12-coated tubes and incubated as described above. After 5 minutes, tubes are gently washed five times with 3 ml of PBS. Cells adherent on the 2G12-coated tubes, i.e. purified GCs, are washed with culture medium (see below) and centrifuged at 600 rpm for 5 minutes. The purified GC suspension is plated at a density of 1000 cells/cm², on 12-mm glass cover slips coated with 50 μg/ml poly-L-lysine and 10 μg/ml laminin. The purified GC are cultured in 400 μl of culture medium, which is a serum-free medium containing Neurobasal (Gibco; Grand Island, N.Y.) with 1 mM glutamine; 10 g/ml gentamicin; B27 supplement (1:50); 40 ng/ml each of human brain-derived neurotrophic factor (BDNF), rat ciliary neurotrophic factor (CNTF), and basic fibroblast growth factor (bFGF); and 5 μM forskolin. Without the added growth factors, virtually all cells die by apoptosis within 2 days. Cultures are maintained at 370 C in humidified atmosphere containing 5% CO2 and 95% air.

### Example 4

### Proteoliposome Incorporation Into Cultured Cells

Retinoblastoma cells. WERI-Rb cells were grown in tissue culture and plated in wells on glass chamber slides as described above. PS I-proteoliposomes, prepared as described above, were introduced into the culture media to final concentrations of between 100 and 400 nmol phospholipid per 10⁶ cells, and the cells were incubated under growth conditions described above. After 30-60 min, the cells were collected by low-speed centrifugation, washed with growth medium, and PS I incorporation was determined by fluorescence analysis (Kruip, J. et al., J. Biol. Chem., 274:18181-18188, 1999) or alternatively by light-induced P700 absorption spectroscopy.

Purified GC cultures. PS I-proteoliposomes are introduced into the culture media to final concentrations of between 100 and 400 nmol phospholipid per 10⁶ cells, and incubated under growth conditions described above for GC cultures. After 30-60 min, the cells are collected by low-speed centrifugation, and washed with growth medium. PS I incorporation is determined as described for retinoblastoma cell cultures.

### Example 5

### Photoreactivity Imparted To Retinoblastoma Cells Treated with PS I-Proteoliposomes

Mammalian cells such as retinoblastoma cells are not normally responsive to light. To demonstrate the efficacy of optical trigger insertion into a retinal cell membrane, cultured Weri-Rb cells were used to study photoreactivity and ion flux in these cells following insertion of PS I reaction centers into their cell membranes.

Experimental Protocol: PS I-proteoliposomes were prepared, with and without incorporated PS I reaction centers, and used for fusion into the membranes of cultured retinoblastoma cells using the procedures described above. Control Weri-Rb cultures were those fused with control liposomes lacking PS I reaction centers. Following addition of liposomes (0.5-2000 ng of phosphorus in 500 μl HBS per well) to the wells, the slides werc incubated for 1-2 h at 37° C. in the dark, wrapped in foil in a light-impermeable box.

To enable measurements of intracellular calcium concentration during photoreactivity testing, the cells were pretreated with a calcium imaging dye, Fluo-3 AM (Molecular Probes, Eugene, Oreg.). Under normal conditions, a cell's interior calcium level is two to three orders of magnitude lower than that of the exterior. Use of the Fluo-3 dye permits assessment of the movement of calcium from the exterior to the interior of a cell, indicated by changes in fluorescence. For Fluo-3 loading, the cells were washed with HBS twice in the dark, then loaded, in the dark, with 16 mM Fluo-3 μM in dimethylsulfoxide (DMSO; Aldrich Chemical, Milwaukee, Wis.), and with 0.02% Pluronic F-127 (Molecular Probes, Eugene, Oreg.)) at 37° C. for 1-2 h. Loading solutions were prepared as follows: Fluo-3 AM (50 mg) was dissolved in 10 ml DMSO to obtain a 5 mM stock solution. Following treatment with Fluo-3, the cells were then washed twice in the dark with 750 μl HBS with or without 1 mM ascorbate. HBS (750 μl) with or without ascorbate was then added.

Fluorometric imaging techniques were used to monitor the intracellular calcium concentrations. The Fluo3-loaded cells were imaged in slide chambers using a Nikon inverted microscope with fluorescence capabilities. Fluorescent response was excited by attenuated light at a wavelength of 490 nm. Fluo-3 fluorescence emission was measured at 510 nm by an intensified charge cooled device (CCD) (Quantix, Photometrics). The signal was collected for 4000 see after individual light pulses at 2 sec lapses with a 10 msec interval, and the collected data were subsequently processed.

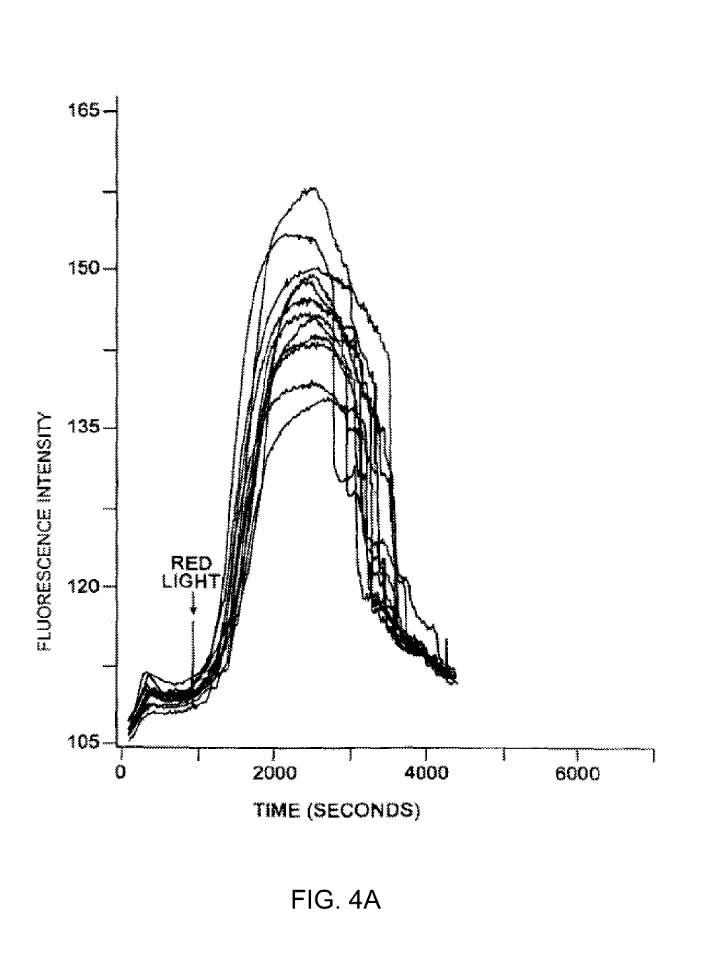

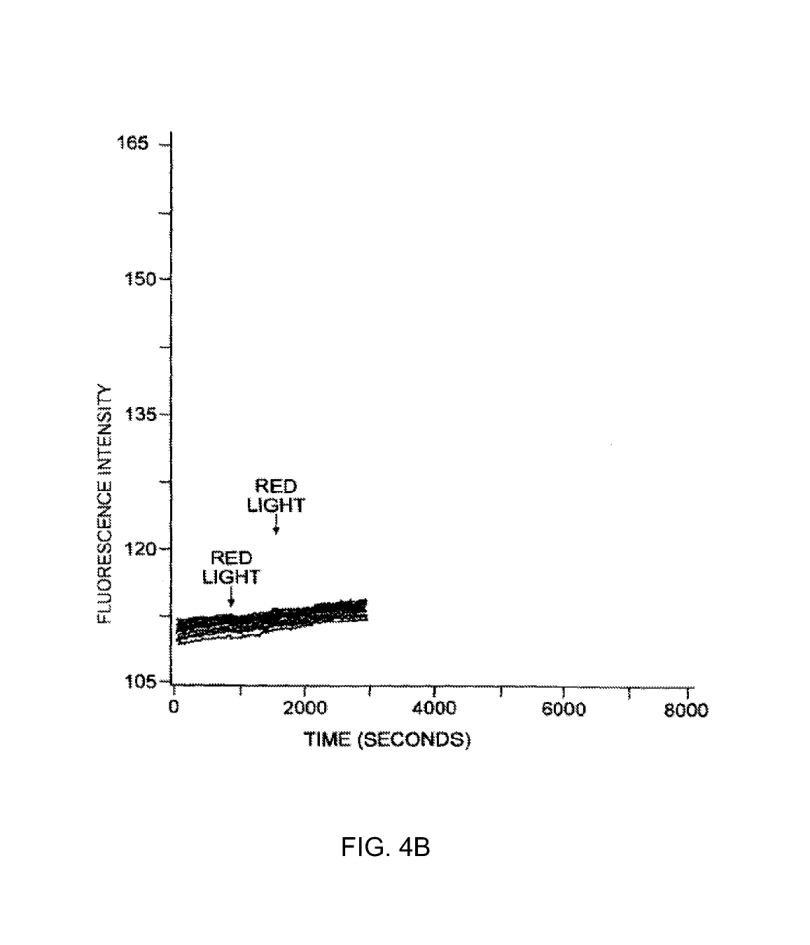

Results. Referring now to FIG. 4, the results are shown from an illumination experiment using cultured Weri-Rb cells treated with PS I-proteoliposmes (FIG. 4A) and with control proteoliposomes (FIG. 4B). Each line on the graphs represents data from a separate cell on the plate. Following stimulation with red light, the PS I-proteoliposome treated cells displayed a marked increase in fluorescence, indicating rapid movement of calcium from the exterior to the interior of the cells (FIG. 4A). By contrast, no such increase was observed in the control cultures treated with proteoliposomes alone (FIG. 4B). These results demonstrated that Weri-Rb cells became photoreactive by the insertion of PS I reaction centers into their plasma membranes. Furthermore, in response to stimulation by light, an influx of ions (in this case calcium) occurred in the cells containing PS I reaction centers within their plasma membranes. Ionic currents across cell membranes are the basic biochemical phenomena associated with the generation of neural signals, or action potentials. The results of this study are consistent with the use of PS I reaction centers for generation of optically-triggered action potentials in retinal cells having PS I reaction centers inserted into their plasma membranes.

### OTHER EMBODIMENTS

While the above specification contains many specifics, these should not be construed as limitations on the scope of the invention, but rather as examples of preferred embodiments thereof. Many other variations are possible.

What is claimed is:

1. A method for imparting photoreactivity to a cell, the method comprising incorporating an isolated optical trigger on the surface or in the membrane of the cell.

2. The method of claim 1, wherein the optical trigger is a Photosystem I reaction center.

3. The method of claim 1, wherein the cell is a nerve cell.

4. The method of claim 3, wherein the cell is a retinal ganglion cell.

5. The method of claim 3, wherein the cell is a retinal bipolar cell.

6. The method of claim 3, wherein the cell is a photoreceptor cell.

7. The method of claim 3, wherein the cell is a retinoblastoma cell.

8-23. (canceled)

24. The method of claim 1, wherein the optical trigger is incorporated in a liposome to form a proteoliposome.

25. The method of claim 24, wherein the proteoliposome is conjugated to an antibody.

26. The method of claim 1, wherein exposure of the optical trigger to light induces an electrical potential in the cell.

27. The method of claim 1, wherein the isolated optical trigger is incorporated in the cell in situ.

28. The method of claim 27, wherein the cell is in a subject with a degenerative retinopathy.

29. The method of claim 28, wherein the degenerative retinopathy is macular degeneration.

30. The method of claim 28, wherein the degenerative retinopathy is retinitis pigmentosa.

31. The method of claim 1, wherein the isolated optical trigger is incorporated in the cell in vivo.

32. The method of claim 31, wherein the cell is in a subject with a degenerative retinopathy.

33. The method of claim 32, wherein the degenerative retinopathy is macular degeneration.

34. The method of claim 32, wherein the degenerative retinopathy is retinitis pigmentosa.
